SEQUENCE LISTING (2 sequences)

SEQ ID NO: 1 - DNA, 50 bp, Artificial Sequence
  Synthetic - Primer
  misc_feature (42)..(42): N is A, C, T, or G
  misc_feature (46)..(46): W is A or T
tcgtcggcagcgtcagatgtgtataagagacagcctacgggnggcwgcag

SEQ ID NO: 2 - DNA, 55 bp, Artificial Sequence
  Synthetic - Primer
  misc_feature (41)..(41): H is A, C, or T
  misc_feature (42)..(42): V is A, C, or G
gtctcgtgggctcggagatgtgtataagagacaggactachvgggtatctaatcc

(19) United States
(12) Patent Application Publication
(10) Pub. No.: US 2022/0265738 A1
(43) Pub. Date: Aug. 25, 2022

(54) SALUTARY EFFECTS OF INDIGENOUS PROBIOTICS AND NANOAEROSOL METHODS TO MITIGATE MICROBIAL INFECTIONS INCLUDING COVID 19

(71) Applicant: UNIVERSITY OF SOUTH FLORIDA, Tampa, FL (US)

(72) Inventor: Alya Limayem, Tampa, FL (US)

(21) Appl. No.: 17/670,982

(22) Filed: Feb. 14, 2022

Related U.S. Application Data
(60) Provisional application No. 63/148,958, filed on Feb. 12, 2021.

(51) Int. Cl. A61K 35/747; A61K 9/19; A61K 47/36; A61K 47/69; A61K 47/02

(52) U.S. Cl. CPC A61K 35/747; A61K 9/19; B82Y 5/00; A61K 47/6929; A61K 47/02; A61K 47/36

(57) ABSTRACT

Provided herein are materials and methods for the treatment of conditions associated with microbial infections including viral and bacterial infections in subjects in need thereof. In particular, provided herein are methods and compositions for treating viral infections, and in particular SARS-CoV-2 infections, in a subject in need thereof, where the methods comprise administering therapeutically effective amounts of indigenous lactic acid bacteria, such as the Lactobacillus species as disclosed herein.

### CROSS-REFERENCE TO RELATED APPLICATIONS

This application claims the benefit of U.S. Appl. No. 63/148,958, filed on Feb. 12, 2021, the content of which is incorporated herein by reference in its entirety.

### STATEMENT REGARDING FEDERALLY SPONSORED RESEARCH OR DEVELOPMENT

N/A.

### SEQUENCE LISTING

A Sequence Listing accompanies this application and is submitted as an ASCII text file of the sequence listing named “173738_02441_ST25.txt” which is 1,027 bytes in size and was created on Feb. 14, 2022. The sequence listing is electronically submitted via EFS-Web with the application and is incorporated herein by reference in its entirety.

### BACKGROUND

New infectious diseases including drug resistant bacteria and COVID-19 have emerged in spikes unparalleled in contemporary history. Bacterial and viral pathogens have substantially mutated and evolved resistant mechanisms to inactivate therapeutic compound along with the rate of drug resistance exceeds development of new therapeutics, requiring alternative antimicrobials and nanotherapeutics. To this end, there remains a need in the field for new therapeutic compositions and improved methods including bacteriotherapy involving indigenous strains. The latter are region specific and natural with no human intervention. Indigenous lactic acid bacteria (ILAB) with nanocomponents are also broad spectrum and cost-effective to mitigate infections caused by microbial pathogens and ensure precise medicine in a short and long run.

### SUMMARY OF THE PRESENT DISCLOSURE

Disclosed herein are methods and pharmaceutical compositions for treating or reducing incidence of a viral infection in a subject in need thereof. In some embodiments, the methods include administering to the subject a therapeutically effective concentration of beneficial bacteria, wherein the beneficial bacteria comprise an indigenous Lactobacillus. In some embodiments, the indigenous Lactobacillus comprises one or more of Lactobacillus salivarius, Lactobacillus fermentum, and Lactobacillus plantarum. In some embodiments, the indigenous Lactobacillus is selected from FSD1-D, FSI3-L, FSD4-D, FSC3-L and FSD3-WC. In some embodiments the method comprises administering two or more indigenous Lactobacillus species (e.g., two or more of Lactobacillus salivarius, Lactobacillus fermentum, and Lactobacillus plantarum) after prior administration of bioactive pre-base nanocomponents, setting a stage for an efficient bacteriotherapy.

In some embodiments, the subject is at risk for contracting a viral infection, or the subject is suspected of having or has been diagnosed with a viral infection, such as a viral infection caused by a virus selected from influenza virus, coronavirus, adenovirus, norovirus, rotavirus, and respiratory syncytial virus. In some embodiments, the viral infection is caused by SARS-CoV-2. In some embodiments, the subject has COVID-19.

In some embodiments, administration comprises inhalation, and in some embodiments, the administered indigenous bacteria are lyophilized, live, or are in spore form. In some embodiments, the bacterial are lyophilized. In some embodiments, the bacteria comprise isolates set forth in Table 1.

In some embodiments, the method includes, prior to (e.g., 1 week, 4 days, 4, days, 3 days, 2 days, 1 day, 12 hours, 6 hours, 5, hours, 4 hours, 3 hours, 2 hours 1 hour, 30 minutes, 15 minutes), or simultaneously with the administration of the Lactobacillus bacteria, administering to the subject a composition comprising a chitosan and zinc-oxide nanocomposite (CZNP). In some embodiments, the CZNP is administered via inhalation.

Disclosed herein are pharmaceutical composition for use in a method of treating a bacterial or viral infection comprising a pharmaceutically effective amount of one or more indigenous Lactobacillus and a pharmaceutically acceptable carrier. In some embodiments, the indigenous Lactobacillus comprises one or more of Lactobacillus salivarius, Lactobacillus fermentum, and Lactobacillus plantarum. In some embodiments, the indigenous Lactobacillus is selected from FSD1-D, FSI3-L, FSD4-D, FSC3-L and FSD3-WC. In some embodiments, the composition comprises two or more indigenous Lactobacillus species, for example, two or more of Lactobacillus salivarius, Lactobacillus fermentum, and Lactobacillus plantarum.

In some embodiments, the pharmaceutical composition is formulated for administration by inhalation, and in some embodiments, the indigenous Lactobacillus are lyophilized, live, or are in spore form. In some embodiments, the bacteria are lyophilized. In some embodiments, the bacteria comprise isolates set forth in Table 1.

In some embodiments, a probiotic composition comprising one or more bacterial isolates set forth in Table 1, and optionally, one or more additional probiotic organisms that enhance the probiotic activity of the bacterial isolate is provided.

These and other embodiments, aspects, advantages, and features of the present invention will be set forth in the description which follows and will become apparent to those skilled in the art by reference to the following description of the invention and referenced drawings or by practice of the invention. The accompanying drawings illustrate one or more implementations, and these implementations do not necessarily represent the full scope of the invention.

### BRIEF DESCRIPTION OF THE DRAWINGS

The present invention will be better understood and features, aspects and advantages other than those set forth above will become apparent when consideration is given to the following detailed description thereof. Such detailed description makes reference to the following drawings, where:

FIG. 1 presents images (EVOS® FL) of biofilm growth of FSI1-D (top left), FSI3-D (top right), FSD1-D (bottom left) and FSC3-LBC (bottom right) at 40×/10× magnification.

FIGS. 2A-2B. (A) CZNPs nanostructure visualized with TEM at 60,000× magnification, measuring 376.78 nm in diameter, demonstrating the encapsulation of multiple electron-dense ZnO nanoparticles by amorphous chitosan. (B) Multiple CZNPs with less electron dense chitosan encapsulating various amounts of darker, more electron dense ZnO, ranging in size from 360 nm to 400 nm, visualized at 30,000× magnification.

FIGS. 3A-3B. (A) CZNPs-mannitol microspheres visualized with TEM at 50,000× magnification, measuring approximately 0.7 μm in diameter, demonstrating electron-dense particles encapsulated by mannitol. (B) CZNPs-mannitol microsphere visualized with TEM at 150,000× magnification, measuring approximately 1 μm in diameter, with several smaller encapsulated particles measuring approximately 100 nm in diameter. The smaller encapsulated particles most likely represent CZNPs components that have been encapsulated by mannitol to form a microsphere formation.

FIGS. 4A-4B. (A) CZNPs-lactose microsphere solution visualized with TEM at 25,000× magnification, measuring between approximately 0.1-0.35 μm in diameter. (B) CZNPs-lactose microsphere visualized with TEM at 40,000× magnification, measuring approximately 2 μm in diameter, with several smaller encapsulated particles measuring approximately 100 nm in diameter. Based on previous trials, the smaller particles measuring between 0.1-0.35 μm appear to be CZNPs components, which are encapsulated by lactose in (B).

FIGS. 5A-5B. (A) CZNPs-mannitol-lactose microspheres visualized with TEM at 15,000× magnification, measuring approximately 1 μm in diameter. (B) CZNPs-mannitol microsphere visualized with TEM at 120,000× magnification; there are several layers, in which measurement from the outer-most layer reveals a diameter of approximately 1 μm, whereas the second layer measures a diameter of approximately 0.75 μm. Finally, the innermost sphere is not completely encapsulated by the second layer, and measures a diameter of approximately 0.3 μm. Based on the size gradients, it is possible that the inner-most sphere is CZNPs, which is then surrounded by mannitol and further encapsulated by lactose.

FIGS. 6A-6C. (A) Untreated heat-inactivated SARS-CoV-2 virus structure measuring 119.79 nm in diameter, visualized with uranyl acetate negative stain using TEM at 250,000× magnification, with protein spikes attached to the viral envelope. (B) A cluster of untreated heat-inactivated SARS-CoV-2 virus particles visualized using TEM at 50,000× magnification. The overall diameter of the cluster measures approximately 900 nm, while a projection of a single virus particle from the cluster measures approximately 170 nm. (C) Multiple untreated heat-inactivated SARS-CoV-2 virus particles visualized using TEM at 50,000× magnification.

FIGS. 7A-7D. (A) Chitosan enveloping the heat-inactivated SARS-CoV-2 virus particles, measuring approximately 230 nm, seen next to separated protein viral spikes measuring approximately 30 nm in diameter, visualized at 300,000×. (B) Treated heat-inactivated SARS-CoV-2 virus clusters measuring approximately 400 nm in diameter, visualized with uranyl acetate negative stain using TEM at 80,000× magnification. The previously polygonal shape has been disrupted and spread out when treated with CZNPs. (C) Multiple treated heat-inactivated SARS-CoV-2 virus particles visualized using TEM at 50,000× magnification; the borders of the treated virus particles are more blurred than seen in untreated particles (compare with FIG. 3C). (D) separated viral protein spikes, visualized at 120,000×, measuring approximately 28 nm in diameter, which were previously seen attached to the untreated SARS-CoV-2 virus (seen in FIG. 3A).

FIGS. 8A-8D. (A & B, top) illustrate the Lactobacillus sp. measuring approximately 2000 nm length and 600 nm in width in average observed at different TEM magnifications, 50,000 and 80,000, respectively. (C & D, bottom), The 2 images elucidate the previously icosahedral shape of the heat-inactivated SARS-CoV-2 virus particles, measuring in average 140 nm have been disrupted and spread out when exposed to Lactobacillus spp including L. casei visualized with TEM at 50,000× magnification. Most of the heat inactivated virus particles were blurred and misshaped in D when compared to the untreated virus. The sizes of SARS-CoV-2 virus can be slightly larger than the theoretical size due to clumping.

### DETAILED DESCRIPTION OF THE PRESENT DISCLOSURE

The methods and compositions provided herein are based, at least in part, on the inventor's development of indigenous probiotics as selective microbial agonists, enhancing both gut integrity and immunity against emerging infectious diseases and multidrug resistant microbes.

In particular, the methods and compositions are based on development of this flora into therapeutic agents for management of viral infections, including COVID-19. Additional strategies, such as a combined immunological approach through a complementary bacteriotherapy is disclosed herein to mitigate a broad spectrum of infectious diseases. Metagenomic sequencing rose to provide not only taxonomic data but the information on the functional interaction between microbes and the host, which therefore should yield a key to the appropriate bacteriotherapy to ensure precise medicine. Without being bound by any particular theory or mode of action, it is believed that lactic acid bacteria (LABs) exert antiviral effects by i) acting as an adsorptive or trapping mechanism by binding to an invading virus, thus inhibiting virus attachment to the host-cell receptor; and/or ii) releasing antiviral agents (e.g., bacteriocins, H₂O₂ and lactic acid) that would disintegrate the lipidic envelope of COVID-19, and creating a dispersal of its protein spikes, denoting viral spread reduction.

Within the last decade, the gut microbiome has been linked to non-communicative disorders such as type 2 diabetes, and immune-related disorders, such as inflammation, allergies, and infectious diseases. Shotgun metagenomic approaches have added important information on microbiome interaction and host physiology allowing greater understanding of the gut microbial community structure enhancing research strategies for effective modulation. A deep understanding of both taxonomy and microbial function suggests novel approaches to immunity reinforcement and long-term protection from emerging diseases. Currently, indigenous probiotics hold promise as selective microbial agonists, enhancing both gut integrity and immunity against emerging infectious diseases.

Accordingly, provided herein are indigenous probiotics including indigenous lactic acid bacteria (ILAB) characterized by their salutary effects on gastrointestinal health. Such salutary effects include, without limitation, improved bile tolerance and decreasing the pH of the gastrointestinal tract, and activity against undesired microorganisms such as pathogenic bacteria (e.g., Salmonella sp., Listeria monocytogenes, Staphyloccocus aureus, Escherichia coli). In some cases, the beneficial bacteria are indigenous lactic acid bacteria (ILAB). Exemplary indigenous lactic acid bacteria are provided in Table 1. For example, the indigenous isolate can be a Lactobacillus isolate including, without limitation, Lactobacillus salivarius, Lactobacillus fermentum, and Lactobacillus plantarum.

As used herein, the term “′indigenous” with reference to bacteria, such as lactic acid bacteria, refer to those bacteria characteristic of, or isolated from, subjects in a particular geographic region, which remain specific to a local population (e.g., a country or a continent). Indigenous lactic acid bacteria (ILAB) comprise natural microflora that reside within and outside the body, in the mucus of our bodies, and comprise cells with no human-made (i.e., engineered) modification.

In some cases, the probiotic isolate exhibits anti-biofilm formation activity, meaning that the probiotic isolate or a composition containing the isolate prevents formation of a biofilm of bacterial, fungal and/or other cells; and/or disrupts and/or eradicates an established or matured biofilm of bacterial, fungal and/or other cells; and/or reduces the rate of buildup of a biofilm of bacterial, fungal and/or other cells on a surface of a substrate.

Any appropriate technique can be used to assay activity of a probiotic isolate of this disclosure against undesired microorganisms. For instance, activity against pathogenic bacteria is often demonstrated using an agar well diffusion assay.

Preferably, indigenous probiotic isolates of this disclosure are characterized by acid tolerance. In some cases, the indigenous probiotic isolates retain essentially the same viability (at least 50% or at least 55%, or at least 60%, or at least 70%, or at least 75%, or at least 80%, or at least 95% CFU) following exposure to acidic conditions relative to viability prior to the test. For instance, acid tolerance is demonstrated in some case by assaying for viability following exposure to acidic conditions (e.g., an acidic culture medium) for a particular length of time. in some cases, viability is assessed prior to, during, and/or following exposure to acidic conditions.

Preferably, indigenous probiotic isolates of this disclosure are further characterized by bile salts tolerance. In some cases, the indigenous probiotic isolates retain essentially the same viability (at least 50%, or at least 55%. or at least 60%, or at least 70%, or at least 75%. or at least 80%, or at least 95% CFU) following exposure to simulated small intestine juices relative to viability prior to the test. In some cases, exposure comprises contacting, simulated small intestine juices (0, 45% bile salts, optionally at pH=8) for a few hours, but other protocols are known in the art.

TABLE 1
ILAB isolates

| Number | Source | Isolate | MALDI-TOF | 16S rRNA gene |
|---|---|---|---|---|
| 1 | Feces of Adult | FSD1-D | Lactobacillus | — |
| | | | salivarius | |
| 2 | Feces of Adult | FSD2-TC | Enterococcus faecium | Enterococcus faecium |
| 3 | Feces of Adult | FSD3-WC | Lactobacillus | Lactobacillus |
| | | | plantarum | plantarum |
| 4 | Feces of Adult | FSD3-LBC | Lactobacillus | Lactobacillus |
| | | | plantarum | rhamnosus |
| 5 | Feces of Adult | FSD3-DT | Lactobacillus | Lactobacillus |
| | | | fermentum | fermentum |
| 6 | Feces of Adult | FSD4-D | Lactobacillus | Lactobacillus |
| | | | fermentum | fermentum |
| 7 | Feces of Adult | FSD4-I | Lactobacillus | * |
| | | | fermentum | |
| 8 | Feces of Child | FSC2-DC | Weissella confusa | Weisella cibaria |
| 9 | Feces of Child | FSC2-L | Pediococcus | Pediococcus |
| | | | pentosaceus | acidilactici |
| 10 | Feces of Child | FSC3-L | Lactobacillus | Lactobacillus |
| | | | fermentum | fermentum |
| 11 | Feces of Child | FSC3-D | Lactobacillus | Lactobacillus |
| | | | fermentum | fermentum |
| 12 | Feces of Child | FSC3-LBC | Lactobacillus | Lactobacillus |
| | | | plantarum | plantarum |
| 13 | Feces of Infant | FSI1-D | Lactobacillus reuteri | Lactobacillus |
| | | | | fermentum |
| 14 | Feces of Infant | FSI2-L | Lactobacillus | Lactobacillus |
| | | | fermentum | fermentum |
| 15 | Feces of Adult | FSI2-DT | Lactobacillus | Lactobacillus |
| | | | fermentum | fermentum |
| 16 | Feces of Infant | FSI2-D | Lactobacillus | Lactobacillus |
| | | | fermentum | fermentum |
| 17 | Feces of Infant | FSI3-D | Lactobacillus | Lactobacillus |
| | | | fermentum | fermentum |
| 18 | Feces of Infant | FSI3-LBC | — | Lactobacillus |
| | | | | fermentum |
| 19 | Feces of Infant | FSI3-L | Lactobacillus | Lactobacillus |
| | | | fermentum | fermentum |
| 20 | Feces of | FSA1-TC | Enterococcus fecium | * |
| | Adolescent | | | |
| 21 | Feces of | FAS1-L | — | * |
| | Adolescent | | | |
| 22 | Breast Milk | BMS5-D | Lactobacillus | Lactobacillus |
| | | | fermentum | fermentum |
| 23 | Breast Milk | BMS5-LBC | Lactobacillus | Lactobacillus |
| | | | fermentum | fermentum |
| 24 | Breast Milk | BMS6-D | — | Lactobacillus |
| | | | | fermentum |
| 25 | Breast Milk | BMS6- | Weissella confusa | Lactobacillus |
| | | DBC | | fermentum |

—unidentified species;
* undetermined

In some cases, indigenous lactic acid bacteria (ILAB) form a probiotic composition comprising one or more bacterial isolates set forth in Table 1. In some cases, it may be advantageous to further include one or more additional probiotic organisms that enhance the probiotic activity of ILAB isolates of the composition.

In another aspect, provided herein is a pharmaceutical composition for use in a method of treating an infection caused by a microorganism such as a viral infection or a bacterial infection. In some cases, the pharmaceutical composition comprises a pharmaceutically effective amount of a bacterial isolate set forth in Table 1 and a pharmaceutically acceptable carrier.

In another aspect, provided herein is a method of treating or reducing incidence of a bacterial or viral infection in a subject in need thereof. As used herein, the term “viral infection” refers to any undesired presence and/or replication of virus in a subject. Such undesired presence of virus may have a negative effect on the host subject's health and well-being. The term “viral infection” encompasses infections involve several species of viral pathogens as well as those that involve a single viral species. In some cases, the viral infection is caused by a virus selected from influenza virus, coronavirus (e.g., a SARS-CoV-2 strain), adenovirus, norovirus, rotavirus, and respiratory syncytial virus.

As used herein, the term “bacterial infection” refers to any undesired presence and/or growth of bacteria in a subject. Such undesired presence of bacteria may have a negative effect on the host subject's health and well-being. While the term “bacterial infection” should not be taken as encompassing the growth and/or presence of bacteria which are normally present in the subject, for example in the digestive tract of the subject, it may encompass the pathological overgrowth of such bacteria. The term “bacterial infection” encompasses infections involve several species of bacterial pathogens as well as those that involve a single bacterial species. Infections involving multiple species of bacterial pathogens are also known as complex, complicated, mixed, dual, secondary, synergistic, concurrent, polymicrobial, or co-infections.

In some cases, the method comprises administering to a subject in need thereof a therapeutically effective concentration of beneficial bacteria, such as the indigenous probiotic bacteria set forth in Table 1. For example, the indigenous isolate can be a Lactobacillus isolate including, without limitation, Lactobacillus salivarius, Lactobacillus fermentum, and Lactobacillus plantarum. As demonstrated herein, isolates FSD1-D, FSI3-L, FSD4-D, FSC3-L and FSD3-WC exhibit high activity against both gram-negative bacteria and gram-positive bacteria. Accordingly, the isolates described herein can be used to treat gram-positive and gram-negative bacterial infections.

As used herein, the term “gram-positive bacterial infection” encompasses conditions associated with or resulting from gram-positive bacterial infection. These conditions include gram-positive sepsis and one or more of the conditions associated therewith, including bacteremia, fever, hypotension, shock, metabolic acidosis, disseminated intravascular coagulation and related clotting disorders, anemia, thrombocytopenia, leukopenia, adult respiratory distress syndrome and related pulmonary disorders, renal failure and related renal disorders, hepatobiliary disease and central nervous system disorders. These conditions also include translocation of gram-negative bacteria from the intestines and concomitant release of endotoxin. Gram-positive bacteria include, without limitation, bacteria from the following species: Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, and Corynebacterium.

As used herein, the term “gram-negative bacterial infection” encompasses conditions associated with or resulting from gram-negative bacterial infection. Gram-negative bacterial species include, without limitation, Pseudomonas aeruginosa, Klebsiella spp., Enterobacter spp., Escherichia coli, Citrobacter freundii, Salmonella typhimurium, Yersinia pestis, and Francicella tulernesis.

In some cases, it will be advantageous to administer a composition comprising two or more types of beneficial bacteria. For example, the combination for administration to the subject can comprise two or more types of the indigenous probiotics set forth in Table 1

As used herein, a “subject in need thereof” refers to a subject at risk for contracting an infection caused by a microorganism such as a viral infection or a bacterial infection. The term also encompasses a subject suspected of having or diagnosed as having an infection caused by a microorganism such as a viral infection or a bacterial infection. As used herein, the phrase “in need thereof” indicates the state of the subject, wherein therapeutic or preventative measures are desirable. Such a state can include, but is not limited to, subjects having a disease or condition caused by an infection (e.g., viral infection, bacterial infection).

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition. For purposes of this disclosure, “treating” or “treatment” describes the management and care of a patient for the purpose of combating the disease, condition, or disorder. The terms embrace both preventative, i.e., prophylactic, and palliative treatment. “Treating” includes the administration of a compound of present invention to prevent the onset of the symptoms or complications, alleviating the symptoms or complications, or eliminating the disease, condition, or disorder. The term “treat” and words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods of this disclosure can provide any amount of any level of treatment or prevention of disease in a mammal. Furthermore, the treatment or prevention provided by the inventive method can include treatment or prevention of one or more conditions or symptoms of the disease or disease state, e.g., bacterial or viral infection, being treated or prevented. Also, for purposes herein, “prevention” can encompass delaying the onset of the disease, or a symptom or condition thereof.

In some embodiments, the probiotic microorganism (e.g., a probiotic Lactobacillus species as provided in Table 1) is live in culture, in spore form, or inactivated. In some embodiments, the microorganism is dead or is lyophilized.

The methods of this disclosure may include administering the therapeutic agent using any amount effective for treating the subject. The exact dosage is chosen by the individual physician in view of the patient to be treated. Dosage and administration are adjusted to provide sufficient levels of the active agent(s) or to maintain the desired effect. Additional factors which are taken into account include the severity of the disease state, e.g., extent of the condition, history of the condition; age, weight and gender of the patient; diet, time and frequency of administration; drug combinations; reaction sensitivities; and tolerance/response to therapy. For any active agent, the therapeutically effective dose can be estimated initially either in cell culture assays or in animal models, usually mice, rabbits, dogs, or pigs. The animal model is also used to determine a desirable concentration range and route of administration.

By “subject” or “individual” or “animal” or “patient” is meant any subject, particularly a mammalian subject, for whom diagnosis, prognosis, or therapy is desired. The present invention is generally applied to humans, but one may use the present invention for veterinary purposes. For example, one may wish to treat commercially important farm animals, such as cows, horses, pigs, rabbits, goats, and sheep. One may also wish to treat companion animals, such as cats and dogs.

In some cases, a composition comprising a bacterial isolate as provided herein is administered to a subject by any method that achieves the intended purpose or is deemed appropriate by those of skill in the art. For example, a composition of the present invention can be administered as a pharmaceutical and may be administered systemically or locally via oral or parenteral administration. The term “administration,” as used herein, refers to the introduction of a substance, such as a vaccine, into a subject's body through or by way of a route that does not include the digestive tract. The administration may include subcutaneous administration, intramuscular administration, transcutaneous administration, intradermal administration, intraperitoneal administration, intraocular administration, intranasal administration and intravenous administration, or oral administration. Parenteral administration includes, for example, administration of injections. Such injections include, for example, subcutaneous injections, intramuscular injections, and intraperitoneal injection. In some cases, intravenous injections such as drip infusions, intramuscular injections, intraperitoneal injections, subcutaneous injections, suppositories, enemas, oral tablets, or the like can be selected. Alternatively, administration may be via the alimentary route, by combining with the food, feed or drinking water e.g. as a powder, a liquid, or tablet, or by administration directly into the mouth as a: liquid, a gel, a tablet, or a capsule, or to the anus as a suppository.

The therapeutic compositions of the present disclosure may be formulated for administration through several pathways including inhalation. In some embodiments, a prior bioactive nanoerosol including zinc oxide, silver, and chitosan are recommended as “pre-base” to treat pathogens and build new immune cells before ILAB administration. In some embodiments, the method includes, prior to (e.g., 3 weeks, 2 weeks, 1 week, 4 days, 4, days, 3 days, 2 days, 1 day, 12 hours, 6 hours, 5, hours, 4 hours, 3 hours, 2 hours 1 hour, 30 minutes, 15 minutes), or simultaneously with the administration of the Lactobacillus bacteria, administering to the subject a composition comprising a chitosan and zinc-oxide nanocomposite (CZNP). In some embodiments, the prebase is administered multiple times over the course of 1 or more weeks, or 1 or more days, prior to administering the beneficial bacteria-containing composition. In some embodiments, the composition further comprises silver. In some embodiments, the CZNP (with or without the silver) is administered via inhalation. For nasal or inhalation delivery, the beneficial bacteria-containing compositions of the present disclosure also may be formulated by methods known to those of skill in the art, and may include, for example, sprays, inhalers, vapors, micro- and/or nano-aerosols. Such formulation may include solubilizing, diluting, or dispersing substances, such as saline, preservatives; absorption promoters; and fluorocarbons. Such formulations may contain any of a variety of known aerosol propellants useful for endopulmonary and/or intranasal inhalation administration. In addition, water may be present, with or without any of a variety of cosolvents, surfactants, stabilizers (e.g., antioxidants, chelating agents, inert gases and buffers). Such compositions may be generally filtered and sterilized and may be lyophilized to provide enhanced stability and to improve solubility.

As describe above, therapeutic compositions may be formulated using customized design for several pathways including inhalation delivery as nano-aerosols or micro-aerosols. As used herein, a nano-aerosol refers to an aerosol comprising particle having at least one dimension including size and morphology at a nanoscale, which can be ensured by some typical excipients including lactose or mannitol. Examples of nano-aerosols include, but are not limited to, solid nanoparticles, flakes of nanometer thickness, fibers of nanometer diameter, nano-shells, gas-phase solutions of macromolecules, coated nanoparticles, binary nanoparticles, and substances having special surfaces, and are sufficiently aerodynamic to flow and reach the targeted host cell. Micro-aerosols as used herein refer to an aerosol comprising particle having at least one dimension at a micro-scale. Examples of micro-aerosols include, but are not limited to, solid micro-particles, flakes of nanometer thickness, fibers of nanometer diameter, micro-shells, gas-phase solutions of macromolecules, coated micro-particles, binary microparticles, and substances having special surfaces and are also sufficiently aerodynamic to flow and reach the targeted host cell. By way of example, in some embodiments, a nano-aerosol or a micro-aerosol comprises one or more indigenous probiotic bacteria, for example as provided in Table 1, and is administered to a subject in need thereof, such as a subject diagnosed with or suspected of having, or at risk of an infection from SARS-CoV-2 virus.

Compositions can be administered to a subject in need thereof in dosage unit form where each discrete dosage unit contains a predetermined quantity of an active ingredient or compound that was calculated to elicit a desirable therapeutic effect when administered with, in some cases, a pharmaceutically acceptable carrier. As used herein, the term “pharmaceutically acceptable carriers” refers to diluents, preservatives, solubilizers, emulsifiers, adjuvants, aqueous and non-aqueous solutions, suspensions, and emulsions. Pharmaceutically acceptable carriers suitable for the pharmaceutical compositions of this disclosure are well known to those skilled in the art and include, without limitation, solvents, diluents, or other liquid vehicles, dispersion or suspension aids, surface active agents, isotonic agents, thickening or emulsifying agents, preservatives, solid binders, and lubricants. Remington's Pharmaceutical Sciences [Ed. by Gennaro, Mack Publishing, Easton, Pa., 1995] describes a variety of different carriers that are used in formulating pharmaceutical compositions and known techniques for the preparation thereof.

A therapeutically effective dose relates to the amount of a compound (or administered microorganism) which is sufficient to improve the symptoms, for example a treatment, healing, prevention or improvement of such conditions. In exemplary embodiments, a therapeutically effective amount or dose is an amount such that one or more of viral load is decreased; viral replication is slowed; symptoms of viral infection are decreased, lessened, or stabilized (i.e., not worsened), as compared to the symptoms in an untreated control subject. Non-limiting examples of symptoms of viral infection (such as infection with SARS-Co-V-2 virus) include fever, shortness of breath, coughing, muscle aches. In an asymptomatic subject, viral load may be decreased, decreased more quickly, or may be observed not to increase as rapidly or to the extent observed in an untreated control.

Therapeutic effectiveness is based on a successful clinical outcome and does not require that the therapeutic agent or agents kill 100% of the organisms involved in the infection. Success depends on achieving a level of antiviral activity that is sufficient to inhibit the virus in a manner that tips the balance in favor of the host. When host defenses are maximally effective, the antiviral effect required may be minimal. Reducing organism load by even one log (a factor of 10) may permit the host's own defenses to control the infection.

In some embodiments, a therapeutically effective dose may comprise between about 1×10¹-1×10³ microorganisms (e.g., ILAB) per dose, 1×10³-1×10²⁰, 1×10⁵-1×10¹⁵ microorganisms per dose; between about 1×10⁶-1×10¹⁴ microorganisms per dose; between about 1×10⁷-1×10¹³ microorganisms per dose; between about 1×10⁸-1×10¹² microorganisms per dose, between about 1×10⁹-1×10¹¹ microorganisms per dose; between about 1×10¹⁰-9×10¹⁰ microorganisms per dose; or about 3×10¹⁰ microorganisms per dose, or less than 10³ microorganisms per dose. In some embodiments, a therapeutically effective dose may comprise about 1×10⁵, 1×10⁶, 1×10⁷, 1×10⁸, 1×10⁹, 1×10¹⁰, 1×10¹², 1×10¹⁵, 1×10²⁰, 1×10²⁵, 1×10³⁰, or 1×10³⁵ microorganisms per dose.

Clinicians, physicians, and other health care professionals can administer a composition to a subject in need thereof according to a method provided herein by a physician or other health professional. In some cases, a single administration of the composition may be sufficient. In other cases, more than one administration of the composition is performed at various intervals (e.g., once per week, twice per week, daily, monthly) or according to any other appropriate treatment regimen. The duration of treatment can be a single dose or periodic multiple doses for as long as administration of a composition provided herein is tolerated by the subject.

Any appropriate method can be practiced determine, detect, or monitor a subject's response to treatment according to a method provided herein. As used herein, “determining a subject's response to treatment” refers to the assessment of the results of a therapy in a subject in response to administration of a composition provided herein or to treatment according to a method provided herein.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The term “comprising” as used herein is synonymous with “including,” “containing,” or “characterized by,” and is inclusive or open-ended and does not exclude additional, unrecited elements or method steps.

The articles “a” and “an” are used herein to refer to one or to more than one (i.e., to at least one) of the grammatical object of the article. By way of example, “an antibody” means one antibody or more than one antibody. As such, the terms “a” (or “an”), “one or more,” and “at least one” are used interchangeably herein.

While the present invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents and alternatives falling within the spirit and scope of the invention as defined by the appended claims.

The compositions and methods of the present disclosure may be more fully understood upon consideration of the following non-limiting Examples.

### EXAMPLES

### Example 1—Salutary Effects of Indigenous Probiotics in Modulating Gut Microbiota to Mitigate Microbial Infections

This example describes 16S metagenomic screening of the indigenous probiotics and assays to determine their antiviral effects. Without being bound by any particular theory or mode of action, it is believed that ILABs exert antiviral effects by i) acting as an adsorptive or trapping mechanism by binding to an invading virus, thus inhibiting virus attachment to the host-cell receptor; or ii) releasing antiviral agents (e.g., bacteriocins, H₂O₂ and lactic acid) that would disintegrate the lipidic envelope of COVID-19, and creating a dispersal of its protein spikes, denoting viral spread reduction.

Of lactic acid producing bacteria (LAB) isolated from the gut, Lactobacillus species are of prime interest to current research studies, due to the health benefits that these strains can provide to the host by inhibiting pathogens from microbiome. Bilateral research studies with colleagues at the West African University of Ghana has generated tremendous data on the isolation of indigenous probiotics. These data revealed that indigenous microbiota, primarily Lactobacillus fermentum and L. rhamnosus, were the most effective antimicrobials in the local population, over the non-indigenous, for treating pathogens (S. aureus, E. coli ATCC 25922, E. coli BAA-2471, S. typhi, and P. aeruginosa) in the gut. A total of 99 indigenous strains were isolated and characterized from human breast milk (n=29) and fecal samples (n=70) using methods ranging from plating growth tests, conforming to (NCCLS) standards and presumptive analysis at species level using MALDI-TOF MS prior to validation through qPCR techniques. The antimicrobial activities of the isolated strains were studied by agar well diffusion assay. In addition, gastrointestinal tolerance and ability to grow biofilms in vitro using 3D Alvetex platform were determined. More than one-third of the identified strains were L. fermentum followed by L. plantarum, L. rhamnosus, L. salivarius, L. reuteri along with E. faecium, Weissella spp. and Pediococcus spp. These isolates were viable at pH 2, 3, and 6, forming biofilms in a 3D Alvetex platform.

Isolation and Identification of Isolates

Several isolates were obtained from breast milk and fecal samples (adults, adolescent, children and infants) on MRSc media. The isolates were gram-stained and analyzed by microscopic observation and the catalase test. Out of 99 colonies isolated, with 29 from breast milk and 70 from fecal samples, 25 of the isolates were gram-positive rods and catalase negative. The 25 gram-positive catalase negative rods were submitted to MALDI-TOF MS and 16S rRNA gene sequencing analysis (Table 1). More than 80% of the isolated species were identified as belonging to the Lactobacillus genus. More than one-third of the species of were identified as L. fermentum. Other species isolated included L. plantarum, L. rhamnosus, L. salivarius, L. reuteri, E. faecium, Weissella spp. and Pediococcus spp. The Jaccard's similarity coefficient between the MALDI-TOF MS and 16S rRNA gene sequencing methods was almost 70%. Isolates 8 and 9 were identified by both methods as Weissella and Pediococcus species respectively. However, the Weissella species was identified as Weissella confusa with MALDI-TOF MS and Weissella cibaria with 16S rRNA gene sequencing, whilst the Pediococcus species was identified as Pediococcus pentosaceus and Pediococcus acidilactici, respectively. Another isolate identified as L. reuteri by MALDI-TOF MS was identified as L. fermentum by 16S rRNA gene sequencing whilst L. plantarum was identified as L. rhamnosus.

Antimicrobial Activity of Isolates

The antimicrobial activities of isolated species were tested against gram-negative P. aeruginosa (ATCC 101145 and local clinical isolate), E. coli (ATCC 25922 and BAA-2471 multi-drug resistant), S. typhi (local clinical isolate) and S. aureus (ATCC 29213). Only 9 out of 25 isolates had activity against both gram-negative and gram-positive tested bacteria (Table 2). Isolates FSD1-D and FSI3-L showed the highest activity against both the gram-negative and gram-positive bacteria, followed by FSD4-D, FSC3-L and FSD3-WC. All tested isolates had activity against P. aeruginosa and E. coli. Only one isolate, FSD4-I, did not show activity against S. typhi. Also, 13 isolates (approximately 60%) did not show a zone of inhibition towards S. aureus.

TABLE 2
Antimicrobial activity of isolates

| | | Antimicrobial activity (zone of inhibition in mm) | | | | | |
|---|---|---|---|---|---|---|---|
| | | | | | E. coli | S. typhi | |
| | | P. aeruginosa | P. aeruginosa | E. coli | (multi-drug | (clinical | S. aureus |
| No | ISOLATE | (clincal isolate) | ATCC 10145 | ATCC 25922 | resistant) | isolate) | ATCC 29213 |
| 1 | FSD1-D | 14.3 ± 3.4 | 7.7 ± 2.1 | 10.3 ± 1.0 | 4.67 ± 0.8 | 14 ± 1.4 | 9.75 ± 2.2 |
| 2 | FSD2-TC | 8.8 ± 3.4 | 7.4 ± 1.3 | 11 ± 0.8 | 5.8 ± 0.9 | 10.6 ± 0.7 | 0 |
| 3 | FSD3-WC | 12.5 ± 3.4 | 8.7 ± 1.2 | 10.5 ± 1.7 | 7.0 ± 0.0 | 13.5 ± 0.7 | 8.5 ± 0.74 |
| 4 | FSD3-LBC | 12.3 ± 4.4 | 8.7 ± 1.2 | 10.5 ± 0.6 | 3.7 ± 1.2 | 12 ± 1.4 | 7 ± 2.64 |
| 5 | FSD3-DT | 10.5 ± 3.7 | 5.3 ± 0.8 | 11.3 ± 0.6 | 4.3 ± 1.5 | 11 ± 1.4 | 0 |
| 6 | FSD4-D | 12.5 ± 4.5 | 7.3 ± 0.6 | 10.6 ± 0.5 | 5.3 ± 0.6 | 12 ± 1.4 | 10.5 ± 0.7 |
| 7 | FSD4-1 | 9.0 ± 0.0 | 9.0 ± 1.0 | 11.3 ± 0.35 | 5.0 ± 1.0 | 0 | 0 |
| 8 | FSC2-DC | 7.7 ± 2.9 | 3.7 ± 2.1 | 11 ± 0.0 | 2.3 ± 1.5 | 10.5 ± 0.7 | 0 |
| 9 | FSC2-L | 9.0 ± 0.0 | 8.3 ± 1.5 | 10.3 ± 1 | 6.0 ± 1.0 | 11.5 ± 0.7 | 0 |
| 10 | FSC3-L | 12.8 ± 3.9 | 7.7 ± 0.6 | 12.5 ± 4.4 | 5.7 ± 0.6 | 10.5 ± 0.7 | 11 ± 1.4 |
| 11 | FSC3-D | 10.3 ± 2.1 | 7.7 ± 1.2 | 11.5 ± 0.6 | 2.3 ± 1.5 | 11.5 ± 0.7 | 0 |
| 12 | FSC3-LBC | 8.8 ± 4.3 | 8.7 ± 0.6 | 13.8 ± 3.3 | 5.3 ± 0.5 | 11.5 ± 0.7 | 10.5 ± 0.7 |
| 13 | FSI1-D | 7.8 ± 3.2 | 9.0 ± 1.0 | 10.6 ± 0.8 | 3.7 ± 0.8 | 10.5 ± 0.7 | 4.25 ± 1.06 |
| 14 | FSI2-L | 10.3 ± 5.1 | 9.0 ± 1.0 | 12.0 ± 2.2 | 6.7 ± 0.6 | 12 ± 0 | 0 |
| 15 | FSI2-DT | 10.4 ± 2.5 | 7.3 ± 1.2 | 10.0 ± 0.8 | 4.7 ± 0.6 | 10.0 ± 0.0 | 0 |
| 16 | FSI2-D | 10.1 ± 3.9 | 8.3 ± 1.2 | 11.4 ± 1.4 | 4.7 ± 1.2 | 13.5 ± 0.7 | 0 |
| 17 | FSI3-D | 7.5 ± 6.1 | 8.3 ± 0.8 | 9.8 ± 1.0 | 3.7 ± 0.6 | 10.5 ± 0.7 | 3.0 ± 4.24 |
| 19 | FSI3-L | 12.3 ± 3.8 | 5.3 ± 0.6 | 12.8 ± 2.9 | 4.0 ± 1.7 | 11 ± 1.4 | 13 ± 1.4 |
| 20 | FSA1-TC | 8 ± 4.6 | * | 11.6 ± 1.0 | * | 11 ± 1.4 | 0 |
| 22 | BMS5-D | 11.8 ± 4.1 | 7.3 ± 0.8 | 11.1 ± 0.9 | 6.3 ± 0.6 | 13.5 ± 2.1 | 0 |
| 23 | BMS5-LBC | 10.5 ± 1.3 | 6.7 ± 0.8 | 11.0 ± 0.8 | 6.7 ± 0.6 | 10.5 ± 0.7 | 0 |
| 25 | BMS5-DBC | 7.5 ± 0.7 | 8.0 ± 1.0 | 10.0 ± 0.0 | 5.7 ± 0.6 | 0 | 0 |

Gastrointestinal Tolerance Assay

The viable counts of selected isolates to pH 1, 2, 3, 6 and 0.3% bile salt concentration are shown in Tables 3 and 4. The data shows maintained cell viability for all studied isolates at pH 2, 3 and 6. However, all isolates lost total viability during the acid test at pH 1 (Table 3).

TABLE 3
Gastric tolerance of selected isolates

| | | pH 1 | | | pH 2 | | | pH 3 |
|---|---|---|---|---|---|---|---|---|
| | Pre-acid | Exposure time | | | | | | |
| Isolate | tolerance | 1 h | 2 h | 3 h | 1 h | 2 h | 3 h | 1 h |
| FSI1-D | 8.21 ± 0.18 | 0 | 0 | 0 | 8.20 ± 0.11 | 8.16 ± 0.17 | 8.25 ± 0.11 | 8.24 ± 0.21 |
| FSI3-D | 7.98 ± 0.03 | 0 | 0 | 0 | 7.81 ± 0.07 | 7.88 ± 0.06 | 7.80 ± 0.10 | 8.01 ± 0.27 |
| FSD1-D | 8.11 ± 0.16 | 0 | 0 | 0 | 8.01 ± 0.05 | 8.01 ± 0.03 | 7.95 ± 0.01 | 8.32 ± 0.24 |
| FSC3-LBC | 8.02 ± 0.24 | 0 | 0 | 0 | 8.14 ± 0.08 | 7.98 ± 0.06 | 7.90 ± 0.10 | 7.90 ± 0.12 |

| | pH 3 | | pH 6 | | |
|---|---|---|---|---|---|
| | Exposure time | | | | |
| Isolate | 2 h | 3 h | 1 h | 2 h | 3 h |
| FSI1-D | 7.94 ± 0.14 | 8.05 ± 0.04 | 7.90 ± 0.12 | 8.18 ± 0.11 | 5.44 ± 0.22 |
| FSI3-D | 8.01 ± 0.07 | 8.09 ± 0.03 | 8.02 ± 0.20 | 8.05 ± 0.14 | 7.98 ± 0.03 |
| FSD1-D | 7.95 ± 0.34 | 8.18 ± 0.21 | 7.94 ± 0.19 | 8.06 ± 0.13 | 8.20 ± 0.14 |
| FSC3-LBC | 8.02 ± 0.20 | 7.94 ± 0.19 | 7.82 ± 0.20 | 7.80 ± 0.18 | 7.93 ± 0.24 |

TABLE 4
Bile salt test

| | Exposure time | | | | |
|---|---|---|---|---|---|
| Isolate | 0 h | 1 h | 2 h | 3 h | 4 h |
| FSI1-D | 8.22 ± 0.09 | 8.27 ± 0.22 | 8.18 ± 0.25 | 7.98 ± 0.03 | 8.25 ± 0.38 |
| FSI3-D | 7.75 ± 0.05 | 7.69 ± 0.36 | 7.92 ± 0.37 | 7.90 ± 0.11 | 7.74 ± 0.17 |
| FSD1-D | 8.39 ± 0.12 | 8.33 ± 0.04 | 8.23 ± 0.24 | 8.23 ± 0.20 | 8.20 ± 0.11 |
| FSC3-LBC | 8.13 ± 0.24 | 7.96 ± 0.06 | 8.13 ± 0.17 | 8.15 ± 0.15 | 8.25 ± 0.22 |

Biofilm Assay

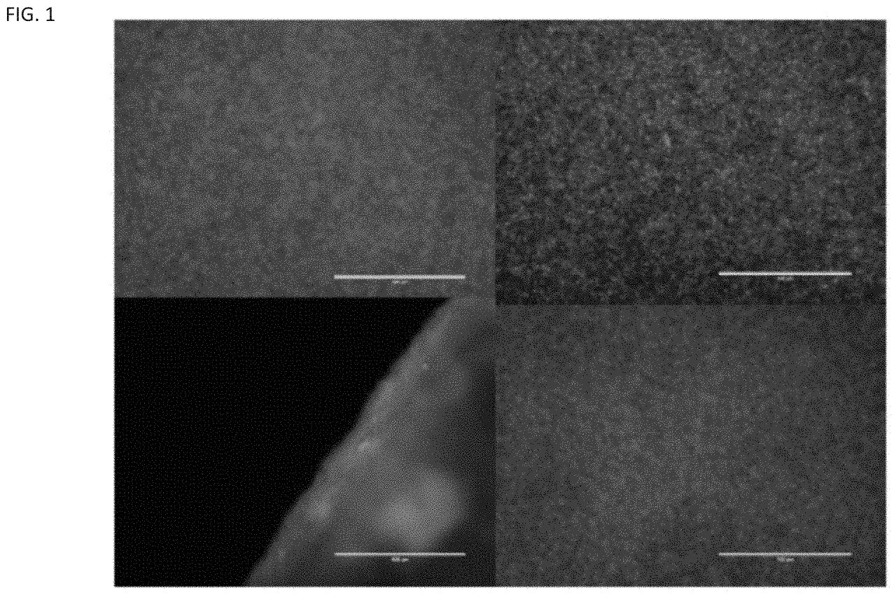

The ability of selected isolates to form biofilm was evaluated using the Alvetex 3D scaffold insert. All tested species formed biofilm on the 3D scaffold (FIG. 1). The biofilm formed for all species comprised of both live (stained green) and dead (stained red) cells. All scaffolds maintained integrity during the period of assay except for scaffold used for culturing isolate FSD1-D.

Experimental Design

Isolation and characterization of the ILAB: Fecal samples were collected from human individuals or rats and stored at −70° C. These samples were thawed and homogenized in 10 mL of phosphate buffered saline (PBS, pH 7.4) using a vortex. A loopful of the fecal suspension was streaked on de Man Rogosa Sharpe (MRS) broth (Oxoid) supplemented with 0.05% w/v L-cysteine hydrochloride and 0.002% w/v of bromophenol blue, and incubated anaerobically at 37° C. for 48 hours. Colonies with different morphological characteristics were picked and streaked on MRS agar supplemented with only 0.05% L-cysteine hydrochloride (MRSc). Pure colonies were identified by MALDI-TOF MS or 16S rRNA gene sequencing and Lactobacillus isolates were selected for further study.

Gastrointestinal (bile) tolerance assay: The selected isolates of Lactobacillus spp. were inoculated (100 μL) of individual strain in 1 mL solution pH adjusted (HCl/NaOH) to 1, 2, 3 and 6 and 0.3% w/v bile salt (Sigma-Aldrich) at 37° C. The viable strains were monitored 1-4 h after incubation.

Samples collection and sequencing through 16S metagenomic analysis: The frozen fecal samples were brought to 37° C. and were analyzed on a 3D Alvetex platform. These samples were supplemented with suitable nutrient media, and experiments were performed in triplicates. A fraction of sample from each well was collected initially and remaining samples were supplemented with indigenous/non-indigenous LAB strains (individual isolates or consortium) to assess how these strains alone or in combination modulate the microbiome. The supplemented samples were collected at different time intervals and stored at −70° C. freezer for further analysis. This study utilized the advanced multiplexed high-throughput Illumina 16S rRNA gene sequencing technique for quantification of the bacterial population at the species level. The DNA was extracted and quantified using Quantifluor dsDNA System (Promega, Madison, Wis.) and was sequenced prior to 16S rRNA analysis. The purity and yield of the extracted DNA was checked using the ND-1000 NanoDrop spectrophotometer (Fisher Scientific, Pittsburgh, Pa.). Primers were selected to target the V3 and V4 regions of the 16S rRNA genes (Table 5).

TABLE 5
Selected Primers to target V3 and V4
regions of the 16S rRNA genes

| | |
|---|---|
| Primer | 5′-TCGTCGGCAGCGTCAGATG |
| | TGTATAAGAGACAGCCTACGGG |
| | NGGCWGCAG-3′ |
| | (SEQ ID NO: 1) |
| Primer | 5′-GTCTCGTGGGCTCGGAGAT |
| | GTGTATAAGAGACAGGACTACH |
| | VGGGTATCTAATCC-3′ |
| | (SEQ ID NO: 2) |

The primers incorporated Illumina adapter sequences, with the 16S-specific sequence portion of primers adopted from Klindworth et al., 2013 [18]. PCR conditions for the 16S rRNA amplification were as follows: an initial denaturation of 95° C. for 3 minutes followed by 25 cycles of denaturation at 95° C. for 30 seconds, annealing at 55° C. for 30 seconds, extension at 72° C. for 30 seconds, and a final extension at 72° C. for 5 minutes. The genomes were sequenced on the Illumina MiSeq System using 600 cycle V3 standard flow cell (Omega Bioservices, Norcross, Ga.). The 300-bp paired end reads for each sample are demultiplexed and quality checked using FastQC 0.11.3. Taxonomies were assigned using UCLUST consensus taxonomy assigner, with a phylogenetic tree built using FastTree 2.1.3 (meta.microbesonline.org/fasttree/ on the World Wide Web). After mapping, the within-community diversity (alpha diversity) was computed for each of the microbial communities, with rarefaction curves generated. Once alpha diversity analysis was finished, between-community diversity (beta diversity) was performed, and Principal Coordinates Analysis (PCoA) plots and distance histograms were generated.

Testing the Effects of the Selected ILAB, Primarily Lactobacillus Spp., on the Dispersal of the COVID-19 Envelope and Spikes with Potential Inactivation of the RNA Viral Genome in a 3D Fiber Platform.

First, transmission electron microscopy (TEM) images are taken of the viral samples to obtain images of the morphological structure of the virus and its surrounding envelope and spikes. Then, 0.05 mL of heat inactivated COVID-19 strain (SARS-CoV-2 ATCC VR-1986HK) is placed onto Alvatex 3D scaffold inserts in a sterile 12-well plate and allowed to stand for 5 minutes. The 3D scaffold inserts mimic the gut collagen-matrix structure. Previously isolated Lactobacillus genus ILABs, are grown in MRSc broth to concentrations ranging from 103 to 108 CFU/mL at 37° C. 0.05 mL of these selected ILAB isolates are inoculated into each well insert infected with the viral strain to create a co-culture. The wells are allowed to stand at room temperature (25° C.) for 3 minutes before being incubated at 37° C. for various periods of time (4 h, 24 h, 48 h). Two control wells are also plated, with the positive control comprising only the viral strain and the negative control consisting of an empty well. Multiple sterile swabs are then taken of the samples for each time period and sent to obtain TEM images for post-treatment analysis, along with the implementation of the artificial intelligence and validated statistics. The above steps are repeated in triplicate for each ILAB isolate used and for each time period measured.

It is anticipated that the SARS-CoV-2 samples treated with ILAB will exhibit dispersal/disintegration of the envelope and spike proteins, and/or that the treated virus will be bound to or trapped by the bacteria. It is further anticipated that the bacterially treated virus samples will exhibit decreased infectivity as compared to untreated virus controls.

It is to be understood that the above description is intended to be illustrative, and not restrictive. The present disclosure has described one or more preferred embodiments, and it should be appreciated that many equivalents, alternatives, variations, and modifications, aside from those expressly stated, are possible and within the scope of the invention. Other embodiments will be apparent to those of skill in the art upon reading and understanding the above description. It should be noted that embodiments discussed in different portions of the description or referred to in different drawings can be combined to form additional embodiments of the present application. The scope should, therefore, be determined with reference to the appended claims, along with the full scope of equivalents to which such claims are entitled.

### Example 2. Pre-Base Nanoaerosols (CZNP) Therapy Prior to Probiotic Treatment for Treatment and Prevention of Infection

Our previously patented nanocomponents, chitosan combined with ZnO (CZNPs) that have proven their effects on multi-drug resistance (MDR) bacteria (see U.S. Pat. Nos. 10,675,301, 10,576,100 and 10,179,146, incorporated herein by reference in their entireties) and is also a promising prophylaxis and a pre-base against virus replication if administrated as a nanoaerosol or ingested. It can act as a virucide in indoor settings and an antiviral that can reach the lungs or the gastrointestinal tract (Gut) microbiome to mitigate the viral proliferation with regard to its safe-use level. Morimoto et al. (2016) reported that rats inhaled ZnO at a maximum concentration of 10 mg/m3 for four weeks with regard to low toxicity. A nanoaerosolized CZNPs (aero-CZNPs) in the form of microspheres, with a diameter between 1 and 5 um, will act as a virucide and once inhaled or ingested, will reach the deep lung including the GIT microbiome, where the carrier carbohydrate will deliver the aero-CZNPs to inhibit viral RNA replication of COVID-19 with no side effects. It is quite possible that a pre-base of CZNPs that is inhaled or ingested prior to probiotic administration will set the stage for the probiotics not only to act as an antimicrobial against viruses including coronavirus with regards to SARS-CoV-2 and other mutating strains but will also repair the damages caused by the microbial infections.

A promising therapeutic agent involves Zn2+ ions for inhibiting RNA polymerase activity in coronavirus and arterivirus families^{1,2,3}. In addition, Zn2+ has been effective in inhibiting respiratory syncytial virus infection-related morbidity in pediatric patients' as well as inhibiting SARS-CoV RNA polymerase⁴. Moreover, ZnO is a source of zinc ions that is widely known and has been FDA approved as being safe⁵. Furthermore, the combination of ZnO with an effective drug delivery vehicle as chitosan^{5,6,7,8} has been proven to be safe to eukaryotic cells with appropriate dosing⁸ and has potential as an antimicrobial agent against the novel coronavirus upon several trials. These nanocomponents, under the safe toxicity threshold, could be nebulized as microspheres to not only directly reach the lungs in a timely manner to treat infected patients and prevent morbidity related to respiratory distress, but also to potentially be used to spray in indoor setting and fomites as a method of prevention and denaturation of virus particles before they are inhaled or ingested. The advantage of administering microspheres through the route of inhalation stems from the ability to localize delivery to the respiratory tract^{9,10,11}, allowing for rapid action and requiring a lower dose than is necessary for systemic delivery, in which the drug may be metabolized by other tissues, and thus fewer and less severe adverse effects 11,12,13,14.

Methods

Preliminary data on the effect of the synergistic chitosan and zinc-oxide nanocomposites (CZNPs) against the structure of heat-inactivated SARS-CoV-2 (ATCC VR-1986HK, BSL1) American Type Culture Collection, Manassas, Va.) the causative agent of COVID-19, was assessed. First, CZNPs were synthesized using methods previously established in our labs, followed by the treatment of the viral strain with CZNPs and subsequent Transmission Electron Microscopy (TEM) imaging.

CZNPs, Probiotic-Pre-Base Synthesis and Efficacy Testing

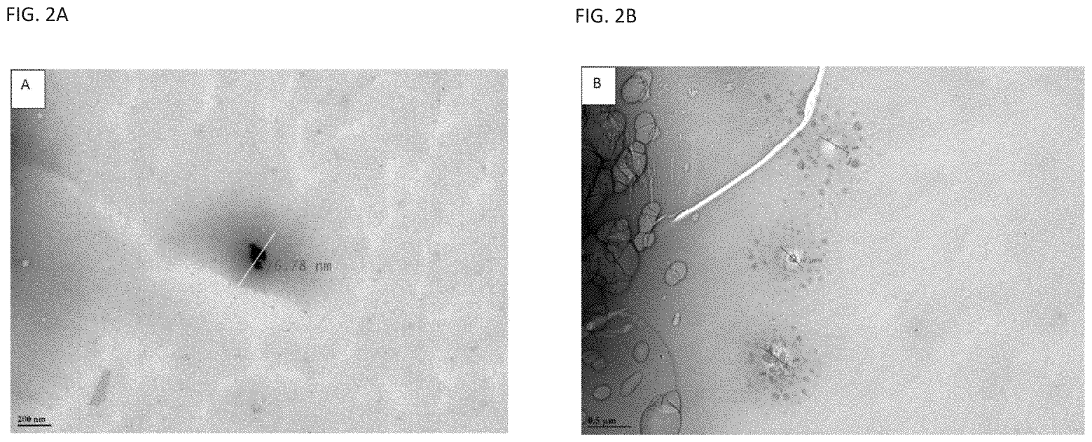

CZNPs nanocomposites were formed prior to treatment of the heat-inactivated SARSCoV-2 virus using a 1:3 ratio (40 mg:120 mg) of ZnO to chitosan nanoparticles. First, ZnO nanopowder (<50 nm; surface area: 54 m2/g; composition: 80.34% zinc and 19.6% oxygen; zeta potential: 9.5-10 mV; acidity/basicity 6.5-7.0; Sigma Aldrich, St. Louis, Mo.) was dissolved in nano-purified water to make solutions of 1.42 mg/ml (40 mg of ZnO in 28 ml of water). The solution was sonicated for at least 3 minutes at an amplitude of 60.0 μm until it was well dispersed. Meanwhile, low molecular weight chitosan (MW: 10 kDa; surface area: 4.56-0.74 m2/gL; surface porosity: 89.3%; zeta potential: 36 mV-40 mV; acidity/basicity 6.2-7.0; 75-85% deacetylated; Sigma Aldrich, St. Louis, Mo.) was dissolved with 15% acetic acid, with heat and stirring for an hour, to make a 10 mg/ml solution. Then, the chitosan solution was adjusted to a pH of approximately 6-7 by adding NaOH dropwise. 28 ml of ZnO solution was mixed with 12 mL of chitosan to make a total volume of 40 ml. The final product was sonicated for 5 minutes until a smooth viscous, gel-like solution was obtained. The product was visualized using TEM (FIGS. 2A-2B).

Preparation of Samples for TEM Imaging

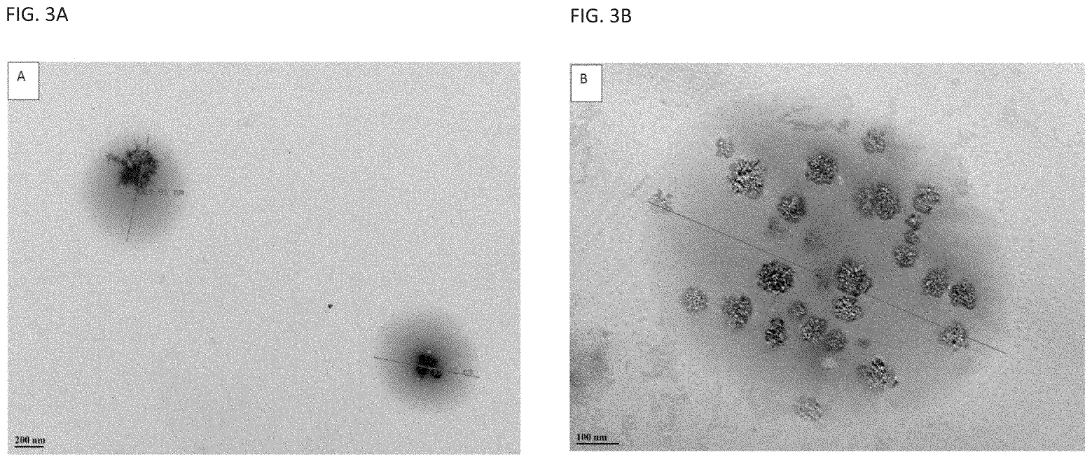

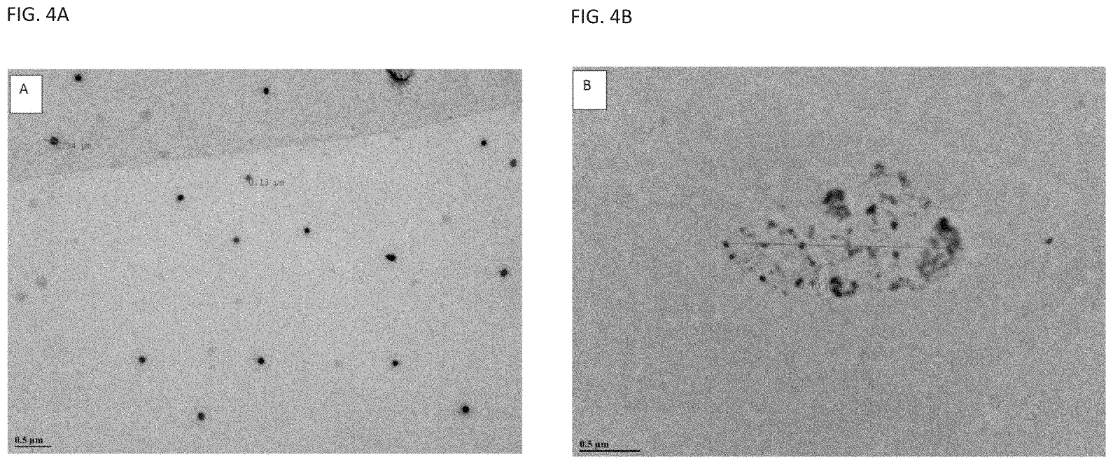

Three samples were selected for imaging via TEM, including a sample with CZNPs alone (FIGS. 2A-2B), a sample with the heat-inactivated SARS-CoV-2 virus alone (FIGS. 3A-3B), and a third sample with equal proportions of both the virus and the CZNPs (FIGS. 4A-4B). Each sample was prepared under BSL-1 level precautions, under a biosafety cabinet; all waste generated was disposed of as biohazardous waste. For each sample, 3 μL of each sample was placed on respective carbon-formvar coated grids. Each sample was allowed to settle on the grid for 2-3 minutes before the excess liquid was wafted with filter paper. Then, 3 μL of uranyl acetate stain (49% concentration) was placed on each grid, acting as a negative stain for the viral envelope. This stain was allowed to settle for an additional 2-3 minutes before the excess was also wafted off. The grids were allowed to dry completely overnight under airtight conditions. The next day, the samples were loaded into the TEM scope for visualization.

Discussion

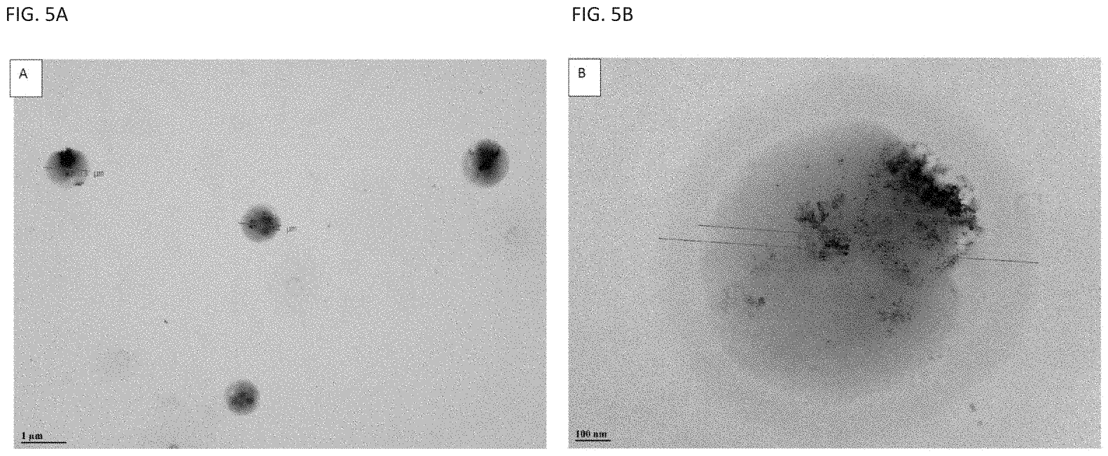

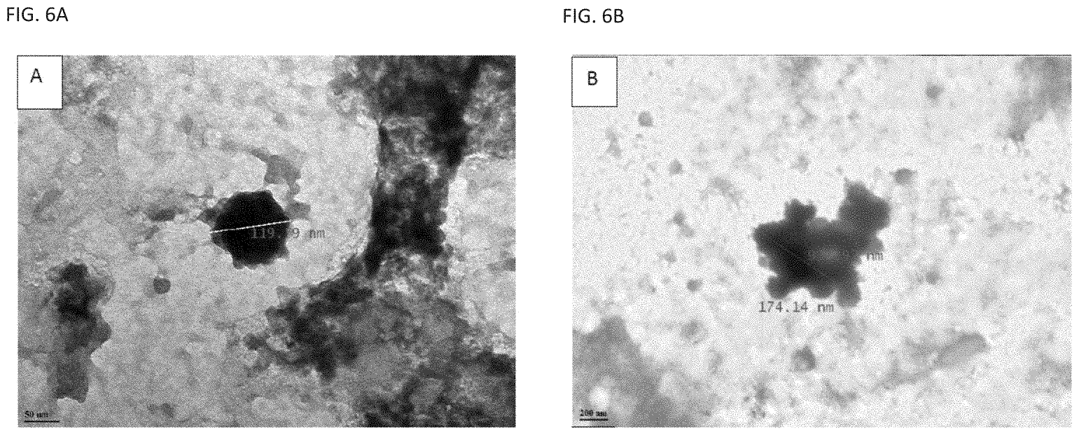

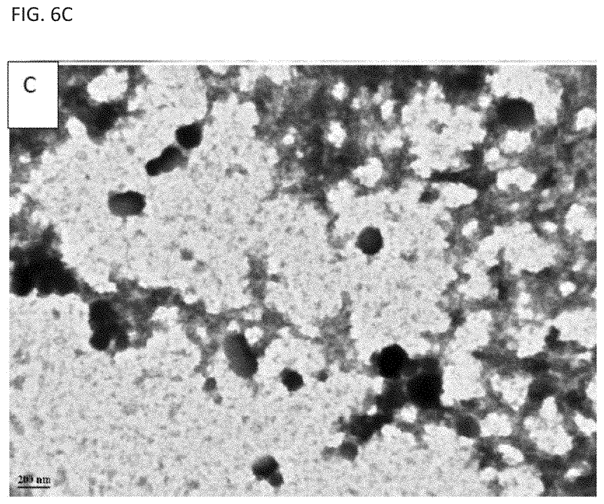

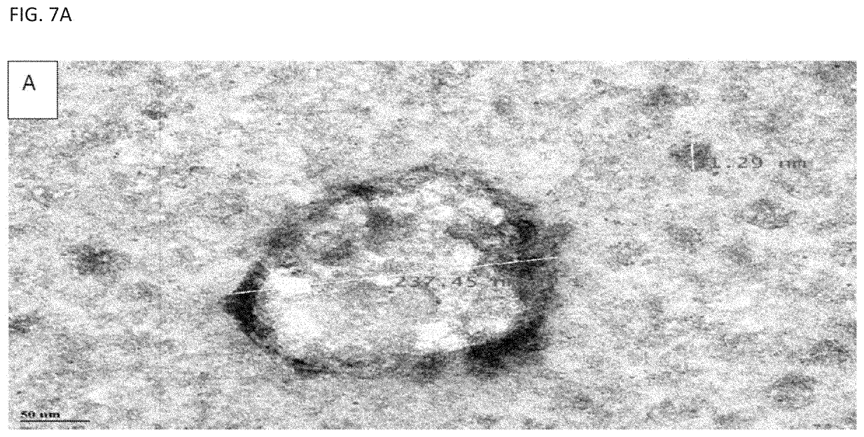

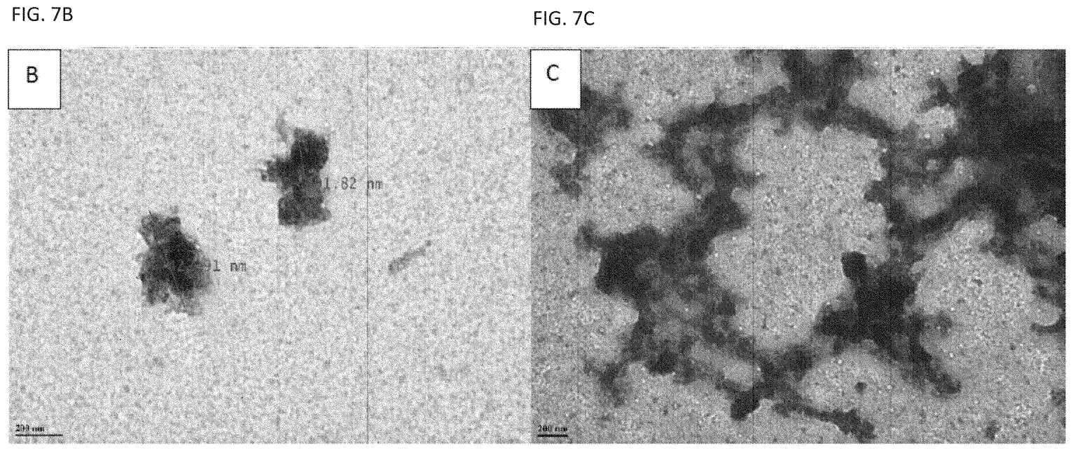

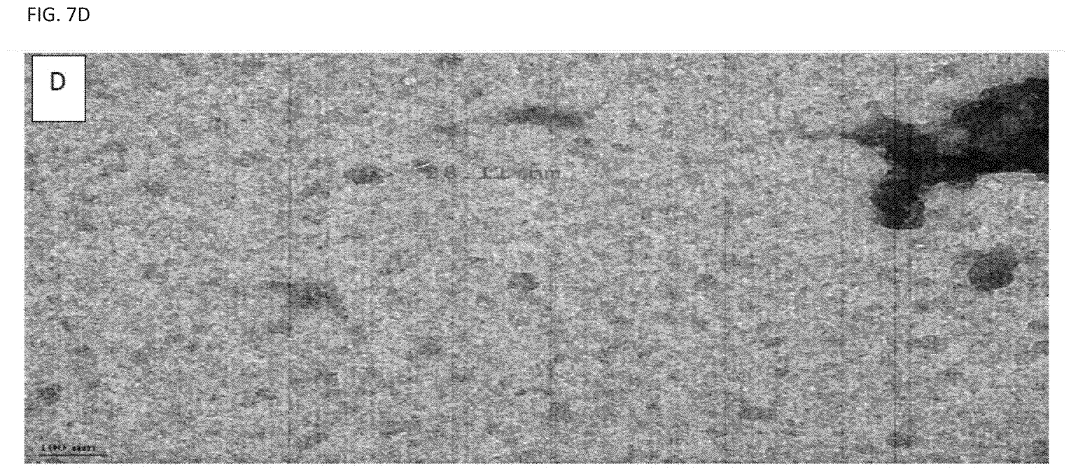

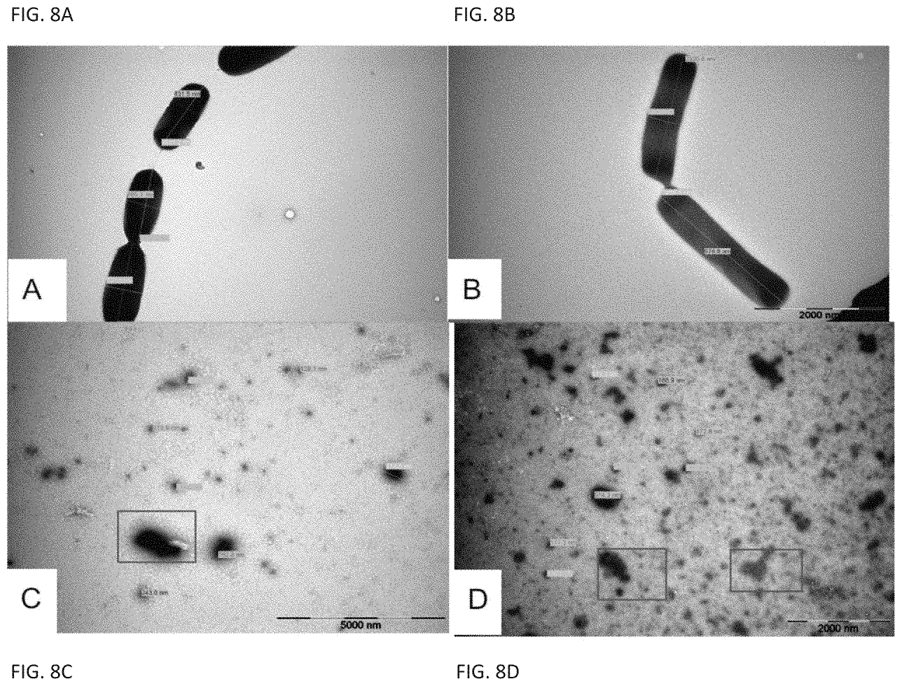

Successful encapsulation of ZnO core by chitosan is visualized in FIG. 2A. The ZnO is seen as a dark core due to its high electron density. Based on the size of the core, which is approximately 150 nm, three ZnO nanoparticles (50 nm each) were encapsulated by chitosan and have agglomerated together. The encapsulating chitosan is represented by slightly less electron dense area surrounding the dark ZnO core. Chitosan is a well-known biopolymer that is effective not only for its low toxicity and scalable attributes but also for its great potential as a nanoparticle drug delivery system, explaining its encapsulating effect on ZnO as seen in the figure. Several CZNPs with less-electron-dense chitosan encapsulating various amounts of darker, more electrondense ZnO, ranging in size from 360 nm to 400 nm, is seen in FIG. 2B. Furthermore, successful microsphere formation of CZNPs with mannitol (FIGS. 3A-3B), lactose (FIGS. 4A-4B) and both mannitol and lactose together (FIGS. 5A-5B) is demonstrated. CZNPsmannitol microspheres (FIGS. 3A-3B) appeared the smallest, ranging from sizes between 0.7 μm to 1 μm, whereas the CZNPs-lactose microspheres were larger, measuring approximately 2 There was no significant difference in size between the CZNPs-mannitol-lactose microspheres (FIGS. 5A-5B) and the CZNPs-mannitol microspheres, as the CZNPs-mannitol-lactose microspheres also measured approximately 1 μm. Of note, however, was that the CZNPs-mannitol-lactose microspheres demonstrated multiple layers of encapsulation (FIG. 5B), which, based on the size gradient, could be the effect of CZNPs (inner sphere; diameter=330 nm) being encapsulated by mannitol (second layer; diameter=0.7 μm) and subsequently by lactose (outer-most layer; diameter=1 μm). In contrast, both the CZNPs-mannitol microsphere (FIG. 3B) and the CZNPslactose microsphere (FIG. 4B) only demonstrated one layer of encapsulation, surrounding several smaller particles averaging 100 nm in diameter, which are presumed to be CZNPs components. A major difference between the CZNPs-mannitol and CZNPs-lactose microspheres, other than the size, is the encapsulation efficiency; several more CZNPs particles were observed to be encapsulated by the mannitol (FIG. 3A) as opposed to the lactose (FIG. 4A), as indicated by the size of the particles. In FIG. 3A, all of the microspheres shown are approximately 0.7 which is larger than the size of CZNPs alone, whereas in FIG. 4A, all of the particles shown appear to have sizes ranging from 0.1-0.35 which is more indicative of CZNPs that are not encapsulated; on the other hand, when CZNPs is encapsulated by the lactose (FIG. 4B), a much greater size (2 μm) is yielded. Thus, much more of CZNPs were visualized being encapsulated by mannitol, rather than lactose. This discrepancy will need to be further studied and statistically validated to ensure it is not due to a natural margin of error, and that there is a difference between encapsulation of CZNPs by mannitol and lactose. Moreover, the difference between the CZNPsmannitol-lactose microspheres and that of CZNPs-mannitol microspheres should be studied to determine whether there is any advantage to using both mannitol and lactose to encapsulate CZNPs. Additionally, successful staining and visualization of SARS-CoV-2 was achieved in FIG. 6. The polygonal shape of the virus is seen in FIG. 6A, which is surrounded by an envelope with multiple protein spikes. As seen by FIG. 6B, multiple virus particles tend to agglomerate together, which could potentially mimic the structure of biofilm from biofilm-forming bacteria; this may be a challenge with regards to treatment. FIG. 6C importantly shows a pretreatment overview of many viral clusters, which can be compared with post-treatment FIG. 7C, which demonstrates blurrier borders and irregular shapes compared to FIG. 6C. This could indicate that upon treatment, CZNPs disrupted the viral envelope, causing the expulsion of the viral RNA, which resulted in blurry borders and irregular shapes. Further studies will need to be done to quantify the effectiveness of CZNPs on viral structure, including the structure of these agglomerated viral particles. In FIG. 7A, chitosan can be seen enveloping the heat-inactivated SARS-CoV-2 virus particles, measuring approximately 230 nm, seen next to separated protein viral spikes measuring approximately 30 nm in diameter. Notably, this chitosan size is smaller than previously seen CZNPs in FIGS. 2A-2B. It could be possible that this chitosan did not envelop ZnO; however it did envelop a cluster of 2 virus particles alone. FIG. 7B, the disrupted structures of treated virus particles are seen; these disrupted structures have become amorphous in shape as opposed to the polygonal untreated viral particle seen in FIG. 6A. Furthermore, protein spikes are absent from the disrupted virus particles, while they were notably present in the untreated sample. Rather than the protein spikes appearing attached to the viral envelope in the treated virus sample, they appear separately as a form of debris, seen in FIG. 7D. This may indicate that CZNPs completely penetrated the viral envelope, allowing for scattering of the protein spikes as debris.

Future experimentation includes formation of a spray containing CZNPs microspheres for better delivery into the respiratory tract, which is where the SARS-CoV-2 strain enters the host body. These microspheres will be characterized with dynamic light scattering (DLS) technique to measure the average size, polydispersity index (PDI) and zeta potential of the formulation. Furthermore, the effect of these CZNPs microspheres on the SARS-CoV-2 strain will be quantified by testing the viricidal potential of the CZNPs microspheres on SARS-CoV-2 infected PPE. Further TEM imaging will be performed, and the CZNPs microspheres will be continually optimized based on the results from trials performed to maximize the effectiveness of CZNPs on inhibiting SARS-CoV-2.

### REFERENCES FOR EXAMPLE 2

1. Te Velthuis, A. J., van den Worm, S. H., Sims, A. C., Baric, R. S., Snijder, E. J., & van Hemert, M. J. (2010). Zn2+ inhibits coronavirus and arterivirus RNA polymerase activity in vitro and zinc ionophores block the replication of these viruses in cell culture. PLoS pathogens, 6(11).
2. Ishida, T. (2019) Review on The Role of Zn2+ Ions in Viral Pathogenesis and the Effect of Zn2+ Ions for Host Cell-Virus Growth Inhibition. American Journal of Biomedical Science and Research, 2(1). DOI: 10.34297/AJBSR.2019.02.000566
3. Suara, Rahaman O. & Crowe Jr, James E. (2004). Effect of Zinc Salts on Respiratory Syncytial Virus Replication. Antimicrobial Agents and Chemotherapy. 48 (3), 783-790.
4. Skalny, A., Rink, L., Ajsuvakova, O., Aschner, M., Gritsenko, V., Alekseenko, S., Svistunov, A. A., Petrakis, D., Spandidos, D. A., Aaseth, J. & Tinkov, A. (2020). Zinc and respiratory tract infections: Perspectives for COVID-19 (Review). International Journal of Molecular Medicine, 46(1), 17-26.
5. Premanathan M, Karthikeyan K, Jeyasubramanian K, Manivannan G. (2011). Selective toxicity of ZnO nanoparticles toward Gram-positive bacteria and cancer cells by apoptosis through lipid peroxidation. Nanomedicine Nanotechnology, Biol Med, 7(2), 184-192.
6. Limayem, A., Gonzalez, F., Micciche, A., Haller, E., Nayak, B. & Mohapatra, S. Molecular identification and nanoremediation of microbial contaminants in algal systems using untreated wastewater. J. Environ. Sci. Heal.—Part B Pestic. Food Contam. Agric. Wastes 51, 868-872 (2016).
7. Limayem, A., Micciche, A., Haller, E., Zhang, C. & Mohapatra, S. Nanotherapeutics for the mutating multi-drug resistant fecal bacteria. J Nanotec Nanosci 1(2):100106 1-13, (2015b).
8. Alonso-Sande, M., Cuna, M., Remunan-Lopez, C., Teijeiro-Osorio, D., Alonso-Lebrero, J. L., & Alonso, M. J. (2006). Formation of new glucomannan-chitosan nanoparticles and study of their ability to associate and deliver proteins. Macromolecules, 39(12), 4152-4158.
9. Zhang, H., Oh, M., Allen, C., & Kumacheva, E. (2004). Monodisperse chitosan nanoparticles for mucosal drug delivery. Biomacromolecules, 5(6), 2461-2468.
10. Mehta, M., Allen-Gipson, D., Mohapatra, S., Kindy, M., & Limayem, A. (2019). Study on the therapeutic index and synergistic effect of Chitosan-zinc oxide nanomicellar composites for drug-resistant bacterial biofilm inhibition. International journal of pharmaceutics, 565, 472-480.
11. Rau, J. L. (2005). The inhalation of drugs: advantages and problems. Respiratory care, 50(3), 367-382.
12. Kuzmov, A., & Minko, T. (2015). Nanotechnology approaches for inhalation treatment of lung diseases. Journal of Controlled Release, 219, 500-518.
13. Buxton D. B. (2009). Nanomedicine for the management of lung and blood diseases. Nanomedicine (London, England), 4(3), 331-339.
14. Grenha, A., Seijo, B., & Remunan-Lopez, C. (2005). Microencapsulated chitosan nanoparticles for lung protein delivery. European journal of pharmaceutical sciences, 25(4-5), 427-437.

### Example 3. Effects of Lactobacillus Ssp. On Viral Infection

Studies have shown that probiotics, specifically Lactobacillus, have positive effects on the immune system and decrease the severity of infections and viruses associated with the upper respiratory tract and gastrointestinal tract. The current SARS CoV-2 has been shown to affect the entire immune system and cause a cytokines storm. The coronavirus protein spikes latch onto healthy cells or through ACE-2 receptors to command or kill the healthy cell (Yang et al., 2020).

Lactobacillus spp. help induce the innate cytokine response that could provide a protective benefit/barrier to COVID-19. A recent study examined Lactobacillus Plantarum (L. Plantarum), AB CD Bifidobacterium longum, and Lactococcus lactis ssp. Lactis and its protective effects against respiratory RNA viruses (Yasunari et al., 2022). The study examined the rebalancing of intestinal microbiomes by using Lactobacillus spp. and other probiotics as a way to control COVID-19 (Yasunari et al., 2022). It was shown that L. Plantarum helped strengthen the host defense mechanism against respiratory viruses and pathogenic influenza viruses. Oral delivery of L. Plantarum in mice helps suppress the virus replication in the lungs and reduce any information in the airway to increase the survival rates. In conjunction with other randomized controlled trials that examined oral intake, L. Plantarum showed a decreased risk of lower and upper respiratory tract infections and decreased respiratory symptoms of infected patients. The IL-6 is crucial as it plays a vital role in the immune dysregulation and systemic hyper information of COVID-19. The use of L. Plantarum is shown to reduce blood IL-6 levels and increase the plasma levels of TNF-α (Yasunari et al., 2022).

Another study examined the use of Lactobacillus rhamnosus GG (LGG) is effective in preventing symptoms of COVID-19 compared to a placebo (Wischmeyer et al., 2022). In this randomized, double-blind, placebo-controlled trial, participants were placed in household contact with somebody who had received a diagnosis of COVID-19 (Wischmeyer et al., 2022). Participants were randomized to either receive a daily oral L. rhamnosus or microcrystalline cellulose placebo for a total of 28 days. 182 participants were enrolled in and randomized clinical trial. It was shown that participants receiving the Lactobacillus had a prolonged onset of symptoms. Out of the 77 symptomatic participants, only 47 underwent testing from a medical provider, and 22 had confirmed COVID-19 laboratory tests. It was determined that there was a trend of decreasing COVID-19 for the participants in the Lactobacillus spp. group. In addition, the microbiome was analyzed by collecting stool samples from the participants. Participants who received the Lactobacillus spp. had a grinder abundance compared to participants who received the placebo. There was a distinct difference between the overall stool microbiota. There was a general change in the participants' microbiota in the Lactobacillus spp. group.

In addition to using specific strains of Lactobacillus, studies have examined altering and manipulating the bio antimicrobial peptides from probiotic bacteria as a drug for COVID-19 (Balmeh et al., 2021). After understanding the RNA strand and using the RNA template of COVID-19, one of the suggested potential therapeutic targets was examining bio-antimicrobial peptides (bio AMPs). In addition, they have been hopeful for other dangerous microorganisms as they produce peptides as an innate immune response to invading pathogens (Balmeh et al., 2021).

In the past, AMPs have been used to treat viral infections such as zika virus, dengue virus, and Influenza A virus (Boas et al., 2019) (Hsieh et al., 2016). One of the AMPs, Lactoferrin, plays an inhibitory role in treating certain viruses. Previous studies have shown that Lactoferrin has had antiviral and inhibitory effects on SARS-CoV-2. In addition to Lactoferrin, it was demonstrated that Baeriocin plantaricin ASM1 peptide (otherwise known as Lactobacillus Plantarum) had the best affinity to SARS-CoV-2 when examining the affinity between the spike protein and ACE2.

A review paper was conducted to demonstrate the effects of probiotics to mitigate against coronavirus disease 2019 (Mirzaei et al., 2021). With the outbreak of SARS-CoV-2, it was said that respiratory infections cause high morbidity and mortality. Viruses include influenza viruses, coronaviruses, respiratory syncytial, parainfluenza viruses, adenoviruses, and rhinoviruses. The findings show the effectiveness of different probiotics in preventing virus-induced respiratory infections. The conclusions were crucial to making inferences and further researching the effects on COVID-19. One of the examined studies showed the immune response and the gut microbiota of using Lactobacillus rhamnosus to reduce the risk of viral respiratory infections in preterm newborns (Luoto et al., 2014). When administering The Lactobacillus, it is shown to have longlasting effects and decrease the impact of rhinovirus infections. In addition, the respiratory symptoms were shorter in children who received the Lactobacillus versus children who did not.

One example showcasing that Lactobacillus can significantly reduce symptoms of infections and provide an innate immunity and barrier against viruses.

In addition to using probiotics, the use of dietary supplements and nutraceuticals have been investigated for the prevention and treatment of COVID-19 (Lordan et al., 2021). This paper examined the use of different vitamins such as Vitamin C, D, omega 3, polyunsaturated fats, probiotics, and zinc to help to increase the immune-boosting effects (all under clinical investigation). Despite much needed further research to back the statements that these vitamins can protect against viruses, it has shown the potential to reduce immunopathology and provide antiviral and anti-inflammatory activities. A study that was covered in this paper examined the use of omega-3 polyunsaturated fatty acids (n-3 PUFA) to prevent or modulate RNA virus infections by amplifying the signaling activity of mitochondrial antiviral-signaling (MAVS) proteins (McCarty & DiNicolantonio, 2020).

### REFERENCES FOR EXAMPLE 3

Balmeh, N., Mahmoudi, S., & Fard, N. A. (2021). Manipulated bio antimicrobial peptides from probiotic bacteria as proposed drugs for COVID-19 disease. Informatics in Medicine Unlocked, 23, 100515. https://doi.org/10.1016/j.imu.2021.100515
Hsieh, I. N., & Hartshorn, K. (2016). The Role of Antimicrobial Peptides in Influenza Virus Infection and Their Potential as Antiviral and Immunomodulatory Therapy. Pharmaceuticals, 9(3), 53. https://doi.org/10.3390/ph9030053
Kageyama, Y., Nishizaki, Y., Aida, K., Yayama, K., Ebisui, T., Akiyama, T., & Nakamura, T. (2021). Lactobacillus plantarum induces innate cytokine responses that potentially provide a protective benefit against COVID-19: A single-arm, double-blind, prospective trial combined with an in vitro cytokine response assay. Experimental and Therapeutic Medicine, 23(1). https://doi.org/10.3892/et.2021.10942
Lordan, R., Rando, H. M., & Greene, C. S. (2021). Dietary Supplements and Nutraceuticals under Investigation for COVID-19 Prevention and Treatment. mSystems, 6(3). https://doi.org/10.1128/msystems.00122-21
Luoto, R., Ruuskanen, O., Waris, M., Kalliomaki, M., Salminen, S., & Isolauri, E. (2014). Prebiotic and probiotic supplementation prevents rhinovirus infections in preterm infants: A randomized, placebo-controlled trial. Journal of Allergy and Clinical Immunology, 133(2), 405-413. https://doi.org/10.1016/j.jaci.2013.08.020
McCarty, M. F., & DiNicolantonio, J. J. (2020). Nutraceuticals have potential for boosting the type 1 interferon response to RNA viruses including influenza and coronavirus. Progress in Cardiovascular Diseases, 63(3), 383-385. https://doi.org/10.1016/j.pead.2020.02.007
Mirzaei, R., Attar, A., Papizadeh, S., Jeda, A. S., Hosseini-Fard, S. R., Jamasbi, E., Kazemi, S., Amerkani, S., Talei, G. R., Moradi, P., Jalalifar, S., Yousefimashouf, R., Hossain, M. A., Keyvani, H., & Karampoor, S. (2021). The emerging role of probiotics as a mitigation strategy against coronavirus disease 2019 (COVID-19). Archives of Virology, 166(7), 1819-1840. https://doi.org/10.1007/s00705-021-05036-8
Vilas Boas, L. C. P., Campos, M. L., Berlanda, R. L. A., de Carvalho Neves, N., & Franco, O. L. (2019). Antiviral peptides as promising therapeutic drugs. Cellular and Molecular Life Sciences, 76(18), 3525-3542. https://doi.org/10.1007/s00018-019-03138-w
Wischmeyer, P. E., Tang, H., Ren, Y., Bohannon, L., Ramirez, Z. E., Andermann, T. M., Messina, J. A., Sung, J. A., Jensen, D., Jung, S. H., Artica, A., Britt, A., Bush, A., Johnson, E., Lew, M. V., Miller, H. M., Pamanes, C. E., Racioppi, A., Zhao, A. T., . . . Sung, A. D. (2022). Daily Lactobacillus Probiotic versus Placebo in COVID-19-Exposed Household Contacts (PROTECT-EHC): A Randomized Clinical Trial. medRXiv. https://doi.org/10.1101/2022.01.04.21268275
Yang, L., Liu, S., Liu, J., Zhang, Z., Wan, X., Huang, B., Chen, Y., & Zhang, Y. (2020). COVID-19: immunopathogenesis and Immunotherapeutics. Signal Transduction and Targeted Therapy, 5(1). https://doi.org/10.1038/s41392-020-00243-2

We claim:

1. A method of treating or reducing incidence of a viral infection in a subject in need thereof, comprising administering to the subject a therapeutically effective amount of beneficial bacteria, wherein the beneficial bacteria comprise an indigenous Lactobacillus.

2. The method of claim 1, wherein the indigenous Lactobacillus comprises one or more of Lactobacillus salivarius, Lactobacillus fermentum, and Lactobacillus plantarum.

3. The method of claim 1, wherein the indigenous Lactobacillus is selected from FSD1-D, FSI3-L, FSD4-D, FSC3-L and FSD3-WC.

4. The method of claim 1, wherein the beneficial bacterial comprise two or more indigenous Lactobacillus species.

5. The method of claim 4, wherein the two or more indigenous Lactobacillus species are selected from the group consisting of Lactobacillus salivarius, Lactobacillus fermentum, and Lactobacillus plantarum.

6. The method of claim 1, wherein the subject is at risk for contracting a viral infection, or the subject is suspected of having or has been diagnosed with a viral infection.

7. The method of claim 6, wherein the viral infection is caused by a virus selected from influenza virus, coronavirus, adenovirus, norovirus, rotavirus, and respiratory syncytial virus.

8. The method of any claim 7, wherein the virus is SARS-CoV-2.

9. The method of claim 1, wherein administration comprises inhalation.

10. The method claim 1, wherein the indigenous bacteria are lyophilized, live, or are in spore form.

11. The method of claim 10, wherein the bacteria are lyophilized.

12. The method of claim 1, wherein the bacteria comprise isolates set forth in Table 1.

13. The method of claim 1, comprising, prior to administration of the Lactobacillus bacteria, administering to the subject a composition comprising a chitosan and zinc-oxide nanocomposite (CZNP).

14. The method of claim 13, wherein the CZNP composition is administered via inhalation.

15. A pharmaceutical composition comprising a pharmaceutically effective amount of one or more indigenous Lactobacillus and a pharmaceutically acceptable carrier.

16. The composition of claim 15, wherein the indigenous Lactobacillus comprises one or more of Lactobacillus salivarius, Lactobacillus fermentum, and Lactobacillus plantarum.

17. The composition of claim 16, wherein the beneficial bacterial comprise two or more indigenous Lactobacillus species selected from the group consisting of Lactobacillus salivarius, Lactobacillus fermentum, and Lactobacillus plantarum.

18. The pharmaceutical composition of claim 15, formulated for administration by inhalation.

19. The composition of claim 15, wherein the one or more one or more indigenous Lactobacillus are lyophilized, live, or are in spore form.

20. A probiotic composition comprising one or more bacterial isolates set forth in Table 1, and optionally, one or more additional probiotic organisms that enhance the probiotic activity of the bacterial isolate.
